(19) United States
(12) Patent Application Publication
(10) Pub. No.: US 2024/0065598 A1
(43) Pub. Date: Feb. 29, 2024

(54) SYSTEM AND METHOD FOR DETECTING NEUROLOGICAL DISORDERS AND FOR MEASURING GENERAL COGNITIVE PERFORMANCE

(71) Applicant: VIEWMIND, INC., New York, NY (US)

(72) Inventor: Gerardo ABEL FERNANDEZ, Bahia Blanca (AR)

(21) Appl. No.: 18/269,302

(22) PCT Filed: Dec. 22, 2021
(86) PCT No.: PCT/US2021/064774

§ 371 Date: Jun. 23, 2023

Related U.S. Application Data
(60) Provisional application No. 63/130,266, filed on Dec. 23, 2020.

(51) Int. Cl. A61B 5/16; A61B 3/11; A61B 5/00

(52) U.S. Cl. CPC A61B 5/165; A61B 3/112; A61B 5/163; A61B 5/7264

(57) ABSTRACT

A system for detecting one or more neurological disorders and/or measuring cognitive performance in a subject by measuring eye movements, oculomotor features or pupil behaviour includes: an eye tracker configured to monitor eye movements of a subject while the subject is visualizing, recognizing, maintaining, controlling, sequencing, following and analyzing targets; a processor configured to receive data from the eye tracker while the subject is visualizing, recognizing, maintaining, controlling, sequencing, following and analyzing the targets; and a display configured to display a test report received from the processor; wherein the processor is further configured to analyze the eye-tracking data for evidence of one or more neurological disorders or general cognitive performance and to report, in the test report, a detection of the one or more neurological disorders or a measure of cognitive performance of the subject.

### CROSS REFERENCE TO RELATED APPLICATION

This application claims the benefit of U.S. Provisional Application Ser. No. 63/130,266, filed Dec. 23, 2020 entitled “SYSTEM AND METHOD FOR DETECTING NEUROLOGICAL DISORDERS AND FOR MEASURING GENERAL COGNITIVE PERFORMANCE WHILE DOING GO AND NO-GO TASK AND MOVING TARGET TASK”. The application is also related to International Application No. PCT/Il2018/051316 (WO 2019/106678), which has an International Filing Date of Nov. 30, 2018, entitled “SYSTEM AND METHOD FOR DETECTING NEUROLOGICAL DISORDERS AND FOR MEASURING GENERAL COGNITIVE PERFORMANCE”, the contents of which are incorporated herein by reference.

### FIELD OF THE INVENTION

The invention relates to systems and methods for detecting neurological disorders and for measuring general cognitive performance, in particular by measuring eye movements, oculomotor features and/or pupil diameter while Go and No-Go task and moving target task.

### BACKGROUND TO THE INVENTION

Using eye tracking as a diagnostic tool has been implemented in the art:

U.S. Pat. No. 4,889,422 discloses an automated system for determining the existence of dyslexia. The system comprises an eye stimulus means, an eye movement detector, a processor that collects data representing eye positions over time, and an analysis program for analyzing the data and categorizing the eye movements into micromovements, saccade movements, pursuit movements, convergent divergent movements, fixations and blinks. If the total number of fixations is greater than the number of visual stimuli, then a first indicator that dyslexia is present is registered.

There is a long felt need for a widely availability tool for diagnosing neurological disorders.

### SUMMARY OF THE INVENTION

It is the object of the present invention to provide a system for detecting one or more neurological disorders in a subject and for measuring also cognitive performance in healthy subjects by registering eye movements while the subject is visualizing, recognizing, maintaining, controlling, inhibiting, sequencing, following and analyzing targets; the system comprising
a. an eye tracker [10], configured to monitor eye movements of a subject [5] while the subject [5] is visualizing, recognizing, maintaining, controlling, inhibiting, sequencing, following and analyzing targets [15];
b. a processor [20], configured to receive data from the eye tracker while the subject [5] is visualizing, recognizing, maintaining, controlling, inhibiting, sequencing, following and analyzing targets [15]; and
c. a display means configured to display a test report received from the processor [20];
wherein the processor is further configured to analyze the eye-tracking data for evidence of one or more neurological disorders and/or general cognitive performance and to report, in the test report [50], a detection of one or more neurological disorders or a measure of cognitive performance of the subject [5].

It is another object of the present invention as described above, wherein the processor is further configured, upon receiving the eye-tracking data from the eye tracker, to:
a. count a total number of ocular fixations of a subject while trying of visualizing, recognizing, maintaining, controlling, inhibiting, sequencing, following and analyzing targets; and
b. if the total number of ocular fixations of a subject when trying of visualizing, recognizing, maintaining, controlling, sequencing, following and analyzing targets is higher than for a control group, then report in the test report that a compromise in attentional processes is detected.

It is another object of the present invention as described above, wherein the processor is further configured, upon receiving the eye-tracking data from the eye tracker, to:
a. count a number of correct landing positions of the subject while visualizing, recognizing, maintaining, controlling, sequencing, following and analyzing targets; and
b. if the percentage number of correct landing positions of the subject is lower than for the control group; then report in the test report that a compromise in executive processes is detected.

It is another object of the present invention as described above, wherein the processor is further configured, upon receiving the eye-tracking data from the eye tracker, to:
a. count a number of correct cue directed outgoing saccades while trying of visualizing, recognizing, maintaining, controlling, sequencing, following and analyzing targets; and
if the percentage number of correct cue directed outgoing saccades that the subject do is lower than for the control group, then report in the test report that a compromise in executive processes is detected. It is another object of the present invention as described above, wherein the processor is further configured, upon receiving the eye-tracking data from the eye tracker, to:
a. count a number of opposite cue directions of outgoing saccades of the subject while trying of visualizing, recognizing, maintaining, controlling, sequencing, following and analyzing targets; and
b. if the percentage number of opposite cue directions of outgoing saccades is higher than for the control group, then report in the test report that a compromise in inhibitory processes is detected.

It is another object of the present invention as described above, wherein the processor is further configured, upon receiving the eye-tracking data from the eye tracker, to:
a. count saccade latency length of the subject while directing sending eyes for visualizing, recognizing, maintaining, controlling, inhibiting, sequencing, following and analyzing targets; and
b. if the saccade latency length (time) is higher than for the control group, then report in the test report that a compromise in speed processing is detected.

It is another object of the present invention to detecting one or more neurological disorders and/or general cognitive performance in a subject by measuring eye movements, wherein the processor is further configured, upon receiving the eye-tracking data from the eye tracker, to
a. compute an average saccade amplitude from one ocular fixation to a next ocular fixation while directing sending eyes for visualizing, recognizing, maintaining, controlling, inhibiting, sequencing, following and analyzing targets; and
b. if the average saccade amplitude is different than for the control group, then report in the test report that a compromise in executive processes is detected.

It is another object of the present invention as described above, further comprising a means for measuring a pupil diameter of the subject, wherein the processor is further configured to
a. track the pupil diameter of the subject when visualizing, recognizing, maintaining, controlling, inhibiting, sequencing, following and analyzing targets; and
b. if the pupil diameter of the subject does not show a modulation as advancing in visualizing, recognizing, maintaining, controlling, inhibiting, sequencing, following and analyzing targets, then report in the test report that that a compromise in noradrenergic system is detected.

It is another object of the present invention as described above, wherein the processor is further configured, upon receiving the eye-tracking data from the eye tracker, to:
a. considering length of fixation duration of the subject while trying of visualizing, recognizing, maintaining, controlling, sequencing, following and analyzing targets; and
b. if the length of fixation duration is higher than for the control group, then report in the test report that a compromise in online processing is detected.

It is another object of the present invention as described above, wherein the processor is further configured, upon receiving the eye-tracking data from the eye tracker, to:
a. considering gaze duration of the subject while trying of visualizing, recognizing, maintaining, controlling, sequencing, following and analyzing targets; and
b. if the length of gaze duration is higher than for the control group, then report in the test report that a compromise in online processing is detected.

It is another object of the present invention to detecting one or more neurological disorders and/or general cognitive performance in a subject by measuring eye movements, wherein the processor is further configured, upon receiving the eye-tracking data from the eye tracker, to
a. count number of correct target recognized while visualizing, recognizing, maintaining, controlling, inhibiting, sequencing, following and analyzing targets; and
b. if the number of correct target recognized is lower than for the control group, then report in the test report that compromises in executive and working memory processes are detected.

It the object of the present invention to provide a system for detecting one or more neurological disorders and to check cognitive performance in a subject by measuring eye movements and pupil behavior and applying an intelligent algorithm; the measuring of eye movements performed while the subject is visualizing, recognizing, maintaining, controlling, inhibiting, sequencing, following and analyzing targets; the system comprising
a. an eye tracker [10], the eye tracker configured to monitor eye movements, oculomotor features and pupil behavior of a subject [5] while the subject [5] is visualizing, recognizing, maintaining, controlling, sequencing, following and analyzing targets [15];
b. a processor [20], the processor configured to receive data from the eye tracker [10] while the subject [5] is visualizing, recognizing, maintaining, controlling, sequencing, following and analyzing targets [15];
c. an intelligent algorithm for learning, identifying, typifying and classifying eye movements features in pathologies and within pathologies; and
d. a display means [40], the display configured to display the output of the intelligent algorithm on a test report received from the processor [20];
wherein the processor is further configured to analyze and modeling the eye-tracking data for evidence of one or more neurological disorders and from cognitive performance and to report, in the test report [50], a detection and classification of the one or more neurological disorders of the subject [5] both, between and within pathologies.

It is another object of the present invention as described above, wherein the processor is further configured, upon receiving the eye-tracking data from the eye tracker, to identify and classifying eye movement features and pupil behavior during visualizing, recognizing, maintaining, controlling, sequencing, following and analyzing targets providing an output of the classifier for reporting in the test report a subject's cognitive performance and/or pathological classification (i.e, the pathology that correspond to the subject because his/her eye movement features; and a value within the pathology (i.e., the level of cognitive, behavioral and biological compromise that the subject shows within a particular pathology).

It is another object of the present invention as described above, wherein the intelligent algorithm is configured to read at least one input, the input selected from a group consisting of:
a. Index of total number of ocular fixations of a subject while visualizing, recognizing, maintaining, controlling, sequencing, following and analyzing targets.
b. Index of correct landing positions of the subject while visualizing, recognizing, maintaining, controlling, sequencing, following and analyzing targets.
c. Index of right cue directed outgoing saccades that the subject do while visualizing, recognizing, maintaining, controlling, sequencing, following and analyzing targets.
d. Index of opposite cue directions of outgoing saccades of the subject while visualizing, recognizing, maintaining, controlling, sequencing, following and analyzing targets.
e. Index of saccade latency length of the subject while directing sending eyes for visualizing, recognizing, maintaining, controlling, sequencing, following and analyzing targets.
f. Average saccade amplitude from one ocular fixation to a next ocular fixation
g. Pupil diameter of the subject while visualizing, recognizing, maintaining, controlling, sequencing, following and analyzing targets
h. Fixation duration of the subject while visualizing, recognizing, maintaining, controlling, sequencing and analyzing targets.
i. Gaze duration of the subjects while visualizing, recognizing, maintaining, controlling, sequencing, following and analyzing targets.
j. Index of blinks coming from the left eye, the right eye or from both eyes.
k. Microsaccades' Factors of Form (FF):
   i. HEWI: shows the micro-saccade's height/width relationship.
   ii. AREA: shows the area of the rectangle in which the micro-saccade is inscribed.
   iii. LONG: is the longitude of the horizontal-vertical plane trajectory of the micro-saccade.
   iv. ANG: is the sum of all the angles in the plane horizontal-vertical plane of the micro-saccade.
   v. AANG: is the sum of all the absolute values of angles in radians in the plane horizontal-vertical plane of the micro-saccade.
   vi. FF gives an estimation of the micro-saccadic trajectory regularity.
   vii. MOD and THETA: are the modulus and the angle of the polar coordinates of the sum of the cartesian coordinates. They give a spatial orientation of the micro-saccade relative to the median of the fixation.
   viii. TIME: is the time duration in milliseconds of the micro-saccade.
   ix. VMIN and VMAX: are the minimum and maximum velocities of the microsaccades in degrees per second.
   x. Micro-saccade rate: is the instantaneous rate in each time bin.
   xi. Directional congruency: is the congruency between the micro-saccade direction and the location of the stimulus.
l. Eye position coming from the left eye, the right eye or from both eyes (i.e., abscissa and ordinate coordinate) during visualizing, recognizing, maintaining, controlling, sequencing, following and analyzing targets.
m. Fixation sequence (i.e., ocular behavior) during visualizing, recognizing, maintaining, controlling, sequencing and analyzing targets. The sequence will be available from images, from matrices, etc.
n. Distance of separation between ocular fixations during visualizing, recognizing, maintaining, controlling, sequencing, following and analyzing targets.
o. Filia information of the subject (i.e., age; years of education; sex; ethnic group; occupation; hours per week of physical activity).
p. Total visualizing, recognizing, maintaining, controlling, sequencing and analyzing targets time (i.e., the time that the subject spent when visualizing targets through a trial).

It is the object of the present invention to provide a method [ ] for evaluating compromises in neurological functions and/or general cognitive resources associated with Go and No-Go tasks, speed processing and moving target task, the method comprising
a. providing a system for evaluating compromises in neurological functions and/or measuring general cognitive resources associated with Go and No-Go tasks, speed processing and moving target task
b. requesting a subject to fixate on a cue reference and/or on a reference target [ ];
c. for a number of repetitions, presenting a stimulus image—cue reference and/or reference target—in one of the places of the screen [ ]; the subject is requested to fixate on a target or avoiding fixating on it; and/or to follow the target as it moves from one place to other one through the screen. The time speed of target's appearance (i.e., how fast the target is presented though the screen) could increase or reduce depending on the subject's velocity for processing targets.
d. repeating steps b and c for a number of trials [ ].

In some embodiments, method [ ] further comprises steps of counting a total number of ocular fixations of a subject while trying of visualizing, recognizing, maintaining, controlling, inhibiting, sequencing, following and analyzing targets; and reporting that a compromise in attentional and executive processes are detected if the total number of ocular fixations of a subject when trying of visualizing, recognizing, maintaining, controlling, sequencing, following and analyzing targets is higher than for a control group [ ].

Physiologically, a compromise in executive processes is correlated with deterioration in the frontal, temporal and/or parietal lobe. In some embodiments, reporting of a pupil behavior abnormality may be used in additional treatment.

In some embodiments, method [ ] further comprises steps of counting a number of correct landing positions of a subject while trying of visualizing, recognizing, maintaining, controlling, inhibiting, sequencing, following and analyzing targets; and reporting that a compromise in executive processes is detected if the percentage number of correct landing positions of the subject is lower than for the control group.

Physiologically, a compromise in executive processes is correlated with deterioration in the frontal, temporal and/or parietal lobe. In some embodiments, reporting of a pupil behavior abnormality may be used in additional treatment.

In some embodiments, method [ ] further comprises steps of counting a number of correct cue directed outgoing saccades of a subject while trying of visualizing, recognizing, maintaining, controlling, inhibiting, sequencing, following and analyzing targets; and reporting that a compromise in executive and exploratory processes are detected if the percentage number of correct cue directed outgoing saccades that the subject do is lower than for the control group [ ].

Physiologically, a compromise in executive processes is correlated with deterioration in the frontal, temporal and/or parietal lobe. In some embodiments, reporting of a pupil behavior abnormality may be used in additional treatment.

In some embodiments, method [ ] further comprises steps of counting saccade latency length of the subject while directing sending eyes for visualizing, recognizing, maintaining, controlling, inhibiting, sequencing, following and analyzing targets; and reporting that a compromise in speed processing are detected if the saccade latency length (time) is higher than for the control group [ ].

Physiologically, a slower saccade speed is correlated with affectation in frontal eye fields, basal ganglia and superior colliculus. In some embodiments, reporting of an affectation in saccade latency may be used in additional treatment.

In some embodiments, method [ ] further comprises steps of counting average saccade amplitude from one ocular fixation to a next ocular fixation for visualizing, recognizing, maintaining, controlling, inhibiting, sequencing, following and analyzing targets; and reporting that a compromise in executive processes is detected if the average saccade amplitude is different than for the control group [ ]

Physiologically, a slower saccade speed is correlated with affectation in basal ganglia and superior colliculus. In some embodiments, reporting of an affectation in saccade latency may be used in additional treatment.

In some embodiments, method [ ] further comprises steps of tracking the pupil diameter while visualizing, recognizing, maintaining, controlling, inhibiting, sequencing, following and analyzing targets; and reporting that a compromise in noradrenergic system is detected if the pupil diameter of the subject does not show a modulation as advancing in visualizing, recognizing, maintaining, controlling, inhibiting, sequencing, following and analyzing targets [ ].

Physiologically, a pupil behavior abnormality is correlated with an affectation in the locus coeruleus, the noradrenergic system and superior colliculus. In some embodiments, reporting of a pupil behavior abnormality may be used in additional treatment.

In some embodiments, method [ ] further comprises steps of counting length of fixation duration of the subject while visualizing, recognizing, maintaining, controlling, inhibiting, sequencing, following and analyzing targets; and reporting that a compromise in online processing is detected if the length of fixation duration is higher than for the control group [ ].

Physiologically, a compromise in online processing is correlated with deterioration in the Prefrontal Cortex, the frontal eye field and in the Dorso-Parietal Cortex. In some embodiments, reporting of a compromise in working memory, inhibition processes and mental flexibility may be used in additional treatment.

In some embodiments, method [ ] further comprises steps of counting length of gaze duration of the subject while visualizing, recognizing, maintaining, controlling, inhibiting, sequencing, following and analyzing targets; and reporting that a compromise in online processing is detected if the length of fixation duration is higher than for the control group [ ].

Physiologically, a compromise in online processing is correlated with deterioration in the Prefrontal Cortex, the frontal eye field and in the Dorso-Parietal Cortex. In some embodiments, reporting of a compromise in working memory, inhibition processes and mental flexibility may be used in additional treatment.

In some embodiments, method [ ] further comprises steps of counting number of correct target recognized of the subject while visualizing, recognizing, maintaining, controlling, inhibiting, sequencing, following and analyzing targets; and reporting that a compromise in working memory is detected if the number of correct target recognized is lower than for the control group [ ]

Physiologically, a compromise in working memory is correlated with an affectation in Prefrontal Cortex and in Posterior Parietal Cortex. In some embodiments, reporting of a compromise in working memory, inhibition processes and mental flexibility may be used in additional treatment.

In some embodiments, method [ ] further comprises steps of counting Eye position coming from the left eye, the right eye or from both eyes target of the subject while visualizing, recognizing, maintaining, controlling, inhibiting, sequencing, following and analyzing targets; and reporting that a compromise in coordination and exploratory processes are detected if the number of eye data is different the control group [ ]

Physiologically, a discrepant eye position is correlated with affectation in frontal eye fields, basal ganglia and superior colliculus and cerebellum. In some embodiments, reporting of an affectation in saccade latency may be used in additional treatment.

In some embodiments, method [ ] further comprises steps of counting Distance of separation between ocular fixations and altered movement coordination of the subject while visualizing, recognizing, maintaining, controlling, inhibiting, sequencing, following and analyzing targets; and reporting that a compromise in coordination and exploratory processes are detected if the number of eye data is different the control group [ ].

Physiologically, a distance between eye fixations and/or altered movement coordination speed is correlated with affectation in frontal eye fields, basal ganglia, precuneus, and superior colliculus. In some embodiments, reporting of an affectation in saccade latency may be used in additional treatment.

### BRIEF DESCRIPTION OF THE FIGURES

FIG. 1 shows a system for detecting one or more neurological disorders of a subject, according to some embodiments of the invention.

### DETAILED DESCRIPTION

In this application, the term “Microsaccades”, also known as “flicks”, are small saccades performed during the fixation periods. They are the largest and fastest of the fixational eye movements. In this application, the term “saccades” relate to quick, simultaneous movement of both eyes between two or more phases of a fixation.

In this application the term “Ocular drift” is the fixational eye movement characterized by a smoother, slower, roaming motion of the eye when fixed on an object.

In this application the term “Ocular microtremors” (OMTs) are small, quick, and synchronized oscillations of the eyes occurring at frequencies in a range of 40 to 100 Hz, although they typically occur at around 90 Hz in the average healthy individual. They are characterized by their high frequency and minuscule amplitude of just a few arcseconds.

In this application the terms “stimulus image” refers to a specific visual pattern or targets presented to the subject in the display. The term “visual task” refers to the activity that performs the subject while processing each stimulus image.

Non-limiting embodiments of the invention are now described in detail.

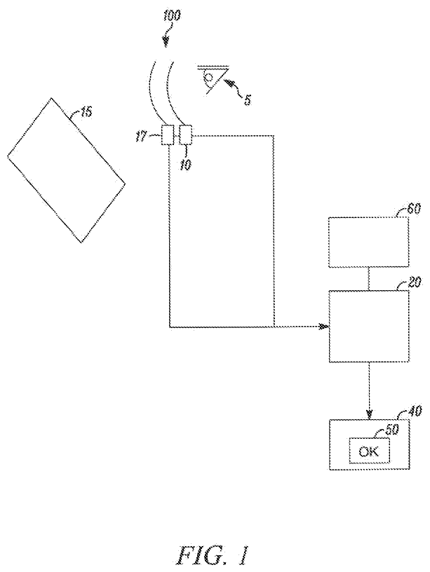

Reference is now made to FIG. 1, showing a system 100 for detecting a neurological disorder or neurological function of a subject 5, according to some embodiments of the invention.

System 100 comprises an eye tracker 10, a means 15 for measuring a pupil diameter, a processor 20, and a display means 40.

Eye tracker 10 can be of any type known in the art; for example, an eye-attached tracker, an optical eye tracker, or an electrooculographic eye tracker.

Means 15 for measuring pupil diameter may comprise, for example, a camera configured to acquire an image of the eye and a processing unit for measuring the pupil diameter from the image. Alternatively to a processing unit, means 15 can comprise a display of the image with manual measurement made while viewing the display.

Eye tracker 10 and means for measuring a pupil diameter 15 are in communicative connection with processor 20. The communicative connections can be of any form(s) known in the art, and can be either wired (e.g., USB, parallel port, or similar) or wireless (e.g. WiFi, Bluetooth, or similar).

Processor 20 receives and executes instructions stored in one or more memory media 60, such as RAM, CD/DVD, HDD, flash memory, and/or any suitable medium. The instructions command processor 20 to: 1) receive eye-tracking data from eye tracker 10; 2) receive pupil diameter data from means 15 of measuring pupil diameter; 3) analyze the eye-tracking and pupil diameter data (further explained herein); 4) report in a test report 50, for display on display means 40, of a detection or non-detection of one or more disorders of memory binding function in subject 5. Display means 40 can be a monitor, a screen of a mobile device such as a smartphone, a printout, or any suitable means of displaying test report 50. Processor 20 may store in memory medium 60 any of the received eye-tracking data, intermediate results at any stage(s) of the analysis, and/or test report 50.

Neurological disorders detected by system 100 can include processing function, such as a compromise in encoding and recognition of targets, a compromise in attentional processes, a compromise in cognitive resources, or any combination thereof. In other embodiments the disorders detected can include Multiple sclerosis (MS), Attention deficit-hyperactive disorder (ADHD), Parkinson disorder (PD), Alzheimer disease (AD), Chronic Stress (CS), Major Depression (MJ), Drug Compromise (DC), etc.

In some embodiments, processor 20 receives eye-tracking data from eye-tracker 10 while subject 5 views each of one or more targets 30. Processor 20 measures gaze durations of subject 5 on each target 30 viewed by subject 5. Processor 20 calculates an average gaze duration on each of the targets 30 by subject 5. If an average of the gaze durations on targets 30 of subject 5 is longer than an average gaze duration for a control group, then processor 20 reports in test report 50 that a compromise in a target encoding and recognition process is detected in subject 5.
a. In some embodiments processor 20 additionally, or alternatively, counts a number of ocular fixations performed by subject 5 while viewing each of the targets 30. If the number of ocular fixations performed by subject 5 while viewing the targets 30 is higher than for a control group, then processor 20 reports in test report 50 that a compromise in an attentional processes is detected in subject 5.

In some embodiments, processor 20 receives pupil diameter data from means 15 of measuring pupil diameter while subject 5 performs activities requiring lower cognitive effort. Processor 20 further receives pupil diameter data from means 15 of measuring pupil diameter while subject 5 performs activities requiring a stronger cognitive effort than for the activities requiring lower cognitive effort. If an average pupil diameter of subject 5 while performing the activities requiring the stronger cognitive effort does not show an increase over an average pupil diameter of subject 5 while performing the activities requiring lower cognitive effort, then processor 20 reports in test report 50 that a compromise in cognitive resources is detected in subject 5.

The control group may comprise a statistically representative cross-section in the same demographic sector as subject 5 (e.g., the same gender, race, national culture, age group, and/or other demographic features of subject 5). Eye-tracking data for the control group may be obtained by system 100 or otherwise gathered from previous research studies and/or clinical studies. Where the average gaze duration or number of ocular fixations of subject 5 is within a selected margin—about one standard deviation of a distribution of the corresponding FIGURE for the control group—of the average FIGURE for the control group, system 100 may treat the average gaze duration or number of ocular fixations of subject 5 as equal to the average corresponding FIGURE for the control group.

It is understood that eye tracking data received by processor 20 may be a series of eyeball positions measured by eye tracker 10, which processor 20 analyzes to find gaze durations and ocular fixations of subject 5. Alternatively, processor 20 may receive a series of pre-processed signals from eye tracker 10, each signaling a gaze duration or that an ocular fixation has occurred. The signals may optionally be accompanied with metadata (e.g., eyeball position, time, and/or length of the ocular fixation).

1. A system for detecting one or more neurological disorders and/or measuring cognitive performance in a subject by measuring eye movements, oculomotor features or pupil behaviour; said measuring of eye movements performed while said subject is visualizing, recognizing, maintaining, controlling, inhibiting, sequencing, following and analyzing targets; said system comprising
a. an eye tracker [10], configured to monitor eye movements of a subject [5] while the subject [5] is visualizing, recognizing, maintaining, controlling, sequencing, following and analyzing targets [15];
b. a processor [20], configured to receive data from said eye tracker while said subject [5] is visualizing, recognizing, maintaining, controlling, sequencing, following and analyzing said targets [15]; and
c. a display means [40] configured to display a test report [50] received from said processor [20];
wherein said processor [20] is further configured to analyze the eye-tracking data for evidence of one or more neurological disorders or general cognitive performance and to report, in said test report [50], a detection of said one or more neurological disorders or a measure of cognitive performance of said subject [5].

2. The system of claim 1, wherein said processor is further configured, upon receiving said eye-tracking data from said eye tracker, to
a. count a total number of ocular fixations of a subject while visualizing, recognizing, maintaining, controlling, sequencing, following and analyzing targets; and
b. if said total number of ocular fixations of a subject when visualizing, recognizing, maintaining, controlling, sequencing, following and analyzing targets is higher than for a control group, then report in said test report that a compromise in attentional processes is detected.

3. The system of claim 1, wherein said processor is further configured, upon receiving said eye-tracking data from said eye tracker, to
a. count a number of correct landing positions of said subject while visualizing, recognizing, maintaining, controlling, following and analyzing said targets; and
b. if said number of correct landing positions of the subject is lower than for the control group; then report in said test report [50] that a compromise in executive processes is detected.

4. The system of claim 1, wherein said processor is further configured, upon receiving said eye-tracking data from said eye tracker, to
a. count a number of right cue directed outgoing saccades while trying of visualizing, recognizing, maintaining, controlling, sequencing, following and analyzing targets; and
b. if said percentage number of right cue directed outgoing saccades that the subject do is lower than for the control group, then report in said test report that a compromise in executive processes is detected.

5. The system of claim 1, wherein said processor is further configured, upon receiving said eye-tracking data from said eye tracker, to
a. count a number of opposite cue directions of outgoing saccades of the subject while trying of visualizing, recognizing, maintaining, controlling, sequencing, following and analyzing targets; and
b. if said percentage number of opposite cue directions of outgoing saccades is higher than for the control group, then report in said test report that a compromise in inhibitory processes is detected.

6. The system of claim 1, wherein said processor is further configured, upon receiving said eye-tracking data from said eye tracker, to
a. compute an average saccade amplitude from one ocular fixation to a next ocular fixation while visualizing, recognizing, maintaining, controlling, sequencing, following and analyzing targets; and
b. if said average saccade amplitude is lower than for said control group, then report in said test report that a compromise in executive processes is detected.

7. The system of claim 1, wherein said processor is further configured, upon receiving said eye-tracking data from said eye tracker, to
a. count saccade latency length of the subject while directing sending eyes for visualizing, recognizing, maintaining, controlling, inhibiting, sequencing, following and analyzing targets; and
b. if said saccade latency length (time) is higher than for the control group, then report in the said test report that a compromise in speed processing is detected.

8. The system of claim 1, further comprising a means [15] for measuring a pupil diameter of said subject, wherein said processor is further configured to
a. track said pupil diameter of said subject when visualizing, recognizing, maintaining, controlling, inhibiting, sequencing, following and analyzing targets; and
b. if said pupil diameter of the subject does not show a modulation as advancing in visualizing, recognizing, maintaining, controlling, inhibiting, sequencing, following and analyzing targets, then report in said test report that that a compromise in noradrenergic system is detected.

9. The system of claim 1, wherein said processor is further configured, upon receiving said eye-tracking data from said eye tracker, to
a. consider length of fixation duration of the subject while trying of visualizing, recognizing, maintaining, controlling, sequencing, following and analyzing targets; and
b. if said the length of fixation duration is longer than for the control group, then report in said test report that a compromise in on-line processing is detected.

10. The system of claim 1, wherein said processor is further configured, upon receiving said eye-tracking data from said eye tracker, to
a. consider gaze duration of the subject while trying of visualizing, recognizing, maintaining, controlling, sequencing, following and analyzing targets; and
b. if said the length of gaze duration is longer than for the control group, then report in said test report that a compromise in on-line processing is detected.

11. The system of claim 1, wherein said processor is further configured, upon receiving said eye-tracking data from said eye tracker, to
a. count number of correct target recognized while visualizing, recognizing, maintaining, controlling, inhibiting, sequencing, following and analyzing targets; and
b. if the number of correct target recognized is lower than for the control group, then report said in the test report that compromises in executive and working memory processes are detected.

12. A system for detecting one or more neurological disorders and to check cognitive performance in a subject by measuring eye movements and pupil behavior and applying an intelligent algorithm; said measuring of eye movements performed while said subject is visualizing, recognizing, maintaining, controlling, inhibiting, sequencing, following and analyzing targets; said system comprising:
a. an eye tracker [10], said eye tracker configured to monitor eye movements and pupil behavior of a subject [5] while the subject [5] is visualizing, recognizing, maintaining, controlling, inhibiting, sequencing, following and analyzing targets [15];
b. a processor [20], said processor configured to receive data from said eye tracker [10] while said subject [5] is visualizing, recognizing, maintaining, controlling, inhibiting, sequencing, following and analyzing targets [15];
c. an intelligent algorithm for learning, identifying, typifying and classifying eye movements features in pathologies and within pathologies; and
d. a display means [40], said display configured to display the output of said intelligent algorithm on a test report [50] received from said processor [20];
wherein said processor [20] is further configured to analyze and modeling the eye-tracking data for evidence of one or more neurological disorders and from cognitive performance and to report, in said test report [50], a detection and classification of said one or more neurological disorders of said subject [5] both, between and within pathologies.

13. The system of claim 8, wherein said processor is further configured, upon receiving said eye-tracking data from said eye tracker, to identify and classifying eye movement features and pupil behavior during visualizing, recognizing, maintaining, controlling, sequencing, following and analyzing targets providing an output of the classifier for reporting in the test report a subject's cognitive performance and/or pathological classification (i.e, the pathology that correspond to the subject because his/her eye movement features; and a value within the pathology (i.e., the level of cognitive, behavioral and biological compromise that the subject shows within a particular pathology).

14. The system of claim 8, wherein said intelligent algorithm is configured to read at least one input, said input selected from a group consisting of:
a. Index of total number of ocular fixations of a subject while visualizing, recognizing, maintaining, controlling, sequencing, following and analyzing targets.
b. Index of correct landing positions of the subject while visualizing, recognizing, maintaining, controlling, sequencing, following and analyzing targets.
c. Index of right cue directed outgoing saccades that the subject do while visualizing, recognizing, maintaining, controlling, sequencing, following and analyzing targets.
d. Index of opposite cue directions of outgoing saccades of the subject while visualizing, recognizing, maintaining, controlling, sequencing, following and analyzing targets.
e. Index of saccade latency length of the subject while directing sending eyes for visualizing, recognizing, maintaining, controlling, sequencing, following and analyzing targets
f. Average saccade amplitude from one ocular fixation to a next ocular fixation while visualizing, recognizing, maintaining, controlling, inhibiting, sequencing, following and analyzing targets
g. Pupil diameter of the subject while visualizing, recognizing, maintaining, controlling, sequencing, following and analyzing targets
h. Fixation duration of the subject while visualizing, recognizing, maintaining, controlling, sequencing, following and analyzing targets.
i. Gaze duration of the subjects while visualizing, recognizing, maintaining, controlling, sequencing, following and analyzing targets.
j. Index of blinks coming from the left eye, the right eye or from both eyes when visualizing, recognizing, maintaining, controlling, inhibiting, sequencing, following and analyzing targets.
k. Microsaccades' Factors of Form (FF):
ki. HEWI: shows the micro-saccade's height/width relationship.
kii. AREA: shows the area of the rectangle in which the micro-saccade is inscribed.
kiii. LONG: is the longitude of the horizontal-vertical plane trajectory of the micro-saccade.
kiv. ANG: is the sum of all the angles in the plane horizontal-vertical plane of the micro-saccade.
kv. AANG: is the sum of all the absolute values of angles in radians in the plane horizontal-vertical plane of the micro-saccade.
kvi. FF gives an estimation of the micro-saccadic trajectory regularity.
kvii. MOD and THETA: are the modulus and the angle of the polar coordinates of the sum of the cartesian coordinates. They give a spatial orientation of the micro-saccade relative to the median of the fixation.
kviii. TIME: is the time duration in milliseconds of the micro-saccade.
kix. VMIN and VMAX: are the minimum and maximum velocities of the microsaccades in degrees per second.
kx. Micro-saccade rate: is the instantaneous rate in each time bin.
kxi. Directional congruency: is the congruency between the micro-saccade direction and the location of the stimulus.
l. Eye position coming from the left eye, the right eye or from both eyes (i.e., abscissa and ordinate coordinate) during visualizing, recognizing, maintaining, controlling, sequencing and analyzing targets.
m. Fixation sequence (i.e., ocular behavior) during visualizing, recognizing, maintaining, controlling, sequencing and analyzing targets. The sequence will be available from images, from matrices, etc.
n. Distance of separation between ocular fixations during visualizing, recognizing, maintaining, controlling, sequencing and analyzing targets.
o. Filia information of the subject (i.e., age; years of education; sex; ethnic group; occupation; hours per week of physical activity).
p. Total visualizing, recognizing, maintaining, controlling, sequencing, following and analyzing targets time (i.e., the time that the subject spent when visualizing targets through a trial).
q. Number of correct target recognized while visualizing, recognizing, maintaining, controlling, inhibiting, sequencing, following and analyzing targets.
